(19) United States
(12) Patent Application Publication
(10) Pub. No.: US 2026/0215732 A1
(43) Pub. Date: Jul. 30, 2026

(54) SMART SPINE IMPLANT FOR ADJACENT LEVEL KINEMATIC AND BIOMECHANICAL ASSESSMENT

(71) Applicant: Warsaw Orthopedic, Inc., Warsaw, IN (US)

(72) Inventors: Arjun Siby Kurian, Memphis, TN (US); Newton H. Metcalf, JR., Memphis, TN (US); Kevin T. Foley, Germantown, TN (US)

(73) Assignee: Warsaw Orthopedic, Inc., Warsaw, IN (US)

(21) Appl. No.: 19/145,382

(22) PCT Filed: Apr. 25, 2024
(86) PCT No.: PCT/IB2024/054039

§ 371 Date: Jul. 2, 2025

Related U.S. Application Data
(60) Provisional application No. 63/498,090, filed on Apr. 25, 2023.

(51) Int. Cl. A61B 5/00

(52) U.S. Cl. CPC A61B 5/4851; A61B 5/0031; A61B 5/4566; A61B 5/7267; A61B 5/7275

(57) ABSTRACT

This document discloses systems and methods related to generating post-operative assessments and/or predictions of spinal disorders. For example, a method includes obtaining one or more first measurements from one or more construct sensors internal to the patient, the one or more first measurements related to a property of a spinal construct of the patient, the spinal construct associated with at least one spinal level. The method further includes obtaining one or more second measurements from one or more implant sensors internal to the patient, the one or more second measurements related to a property of a spinal implant of the patient, the spinal implant associated with a different level than the at least one spinal level of the spinal construct. The method further includes determining, based on the one or more first measurements and the one or more second measurements, a condition of the patient.

This application claims priority from U.S. Provisional Patent Application 63/498,090, filed 25 Apr. 2023, the entire content of which is incorporated herein by reference.

### FIELD

The present disclosure generally relates to a system that use information from multiple sources to generate a post-operative assessment and/or prediction of one or more various spinal disorders.

### BACKGROUND

Proximal Junctional Kyphosis (PJK) and/or Proximal Junctional Failure (PJF) are possible complications after long-segment (e.g., including four or more vertebral levels) instrumented fusion. PJK may be detected in x-ray images indicating that a pathologic problem has already developed around the adjacent segment following a spinal fusion. PJK is not an instantaneous symptom but is considered one of various ongoing adjacent segmental problems. Some PJK patients may display no symptoms whereas others, sometimes referred to as proximal junctional failure (PJF) patients, express clinical symptoms accompanied by pain, walking disturbance, and neurologic deficit, requiring reoperation in some severe cases. PJF can be caused by adjacent disc degeneration, hardware loosening, or fractures at the Uppermost Instrumented Vertebrae (UIV) or in an adjacent vertebrae (UIV+1). Early detection of PJK may allow for intervention before reaching the level of PJF, but frequent and/or ongoing x-ray imaging can be inconvenient, intrusive, costly, and harmful. This document describes methods and systems that are directed to addressing the problems described above, and/or other issues.

### SUMMARY

The systems and techniques of this disclosure generally relate to post-operative monitoring and/or assessment of a patient.

In an example embodiment, a method of generating a post-operative assessment of a patient is disclosed. The method includes obtaining one or more first measurements from one or more construct sensors internal to the patient, the one or more first measurements related to a property of a spinal construct of the patient, the spinal construct associated with at least one spinal level. The method further includes obtaining one or more second measurements from one or more implant sensors internal to the patient, the one or more second measurements related to a property of a spinal implant of the patient, the spinal implant associated with a different level than the at least one spinal level of the spinal construct. The method further includes determining, based on the one or more first measurements and the one or more second measurements, a condition of the patient.

Implementations of the disclosure may include one or more of the following optional features. In some examples, the one or more construct sensors include at least one construct position sensor configured to provide a measurement of the position of the spinal construct. The one or more implant sensors may include at least one implant position sensor configured to provide a measurement of the position of the spinal implant. Determining the condition of the patient may include evaluating one or more biomechanical factors over time, the one or more biomechanical factors determined by the measurement of the at least one construct position sensor and the measurement of the at least one implant position sensor. In some examples, at least one of the one or more biomechanical factors is a range of motion of the spinal implant relative to the spinal construct. Determining the condition of the patient may include applying the one or more first measurements and the one or more second measurements to a machine-learning model trained, via supervised training, to predict the condition of the patient based on the one or more first measurements and the one or more second measurements. In some examples, the machine-learning model is further trained, via supervised training, to recommend a procedure to address the condition and the method further includes applying the one or more first measurements and the one or more second measurements to the machine-learning model, causing the machine-learning model to recommend the procedure. The machine-learning model may be trained to predict Proximal Junction Kyphosis or Proximal Junction Failure. In some examples, the spinal construct includes a multilevel spinal construct and the different level is adjacent to a level of the multilevel spinal construct. In some examples, the one or more construct sensors include at least one strain gauge. In some examples, at least one sensor includes an impedance sensor configured to measure a status of a fusion process. In some examples, the spinal construct includes a multilevel thoracolumbar deformity construct.

In an example embodiment, a post-operative monitoring system is disclosed. The system includes posterior spinal instrumentation associated with two or more vertebrae, the posterior spinal instrumentation including one or more spinal instrumentation sensors configured to measure a property of the posterior spinal instrumentation. The system further includes a spinal implant including one or more spinal implant sensors configured to measure a property of the spinal implant, the spinal implant associated with a different vertebra than the two or more vertebrae of the posterior spinal instrumentation. The system further includes a reader device configured to receive measurement data from the one or more spinal instrumentation sensors and the one or more spinal implant sensors and a patient assessment system configured to determine a condition of a patient based on the measurement data from the one or more spinal instrumentation sensors and the one or more spinal implant sensors.

Implementations of the disclosure may include one or more of the following optional features. The one or more spinal instrumentation sensors may include at least one instrumentation position sensor configured to provide a measurement of the position of the posterior spinal instrumentation. The one or more spinal implant sensors may include at least one implant position sensor configured to provide a measurement of the position of the spinal implant. The patient-assessment system may be configured to determine the condition of the patient by evaluating one or more biomechanical factors over time, the one or more biomechanical factors based on the measurement of the at least one instrumentation position sensor and the measurement of the at least one implant position sensor. In some examples, at least one of the one or more biomechanical factors is a range of motion of the spinal implant relative to the posterior spinal instrumentation. The patient assessment system may be configured to determine the condition of the patient by applying the measurement data from the one or more spinal instrumentation sensors and the one or more spinal implant sensors to a machine-learning model trained, via supervised training, to predict the condition of the patient based on the measurement data from the one or more spinal instrumentation sensors and the one or more spinal implant sensors. In some examples, the machine-learning model is trained to predict Proximal Junction Kyphosis or Proximal Junction Failure. The machine-learning model my be further trained, via supervised training, to recommend a procedure to address the condition and the patient-assessment system may be further configured to recommend the procedure based on the machine-learning model. In some examples, the different vertebra is adjacent to one of the two or more vertebrae. In some examples, the one or more spinal instrumentation sensors include at least one strain gauge. The posterior spinal instrumentation may include a multilevel thoracolumbar deformity construct. In some examples, at least one sensor includes an impedance sensor configured to measure a status of a fusion process.

### BRIEF DESCRIPTION OF DRAWINGS

FIG. 1 illustrates example sensor placement.

FIG. 2 illustrates an example monitoring system.

FIG. 3 illustrates an example machine-learning environment.

FIG. 4 illustrates a flow chart of an example method of training a machine-learning model.

FIG. 5 illustrates a flow chart of an example method of assessing a patient.

FIG. 6 depicts a block diagram of an example of internal hardware that may be used to contain or implement program instructions according to an embodiment.

### DETAILED DESCRIPTION

Embodiments of the present disclosure relate generally, for example, to post-operative monitoring and/or assessment of a patient, e.g., to provide early detection of spinal conditions, such as Proximal Junctional Kyphosis (PJK) and/or Proximal Junctional Failure (PJF) without requiring frequent and/or ongoing x-ray imaging. The exemplary embodiments of the disclosed monitoring/detection system(s) (and related methods of use) are discussed in terms of medical devices and/or implants for the treatment of musculoskeletal disorders and more particularly, in terms of vertebral fixation screws and interbody cages including, for example, pedicle screws (or other form of anchoring assembly), as well as hooks, cross connectors, offset connectors and related systems for use during various spinal procedures or other orthopedic procedures and that may be used in conjunction with other devices and instruments related to spinal treatment, such as rods (or other longitudinal members), wires, plates, intervertebral spacers, and other spinal or orthopedic implants, insertion instruments, and/or methods for treating a spine, such as open procedures, mini-open procedures, or minimally invasive procedures. Exemplary prior art devices that may be used (or may be modified for use) within the scope of this disclosure include, for example, devices disclosed in U.S. Pat. Nos. 6,485,491; 8,057,519; 11,298,162; 11,529,208; 13/182,942; and 11,278,238; and U.S. patent application Ser. Nos. 18/062,867; 18/068,140; and 18/183,484. The disclosures of these patents and patent applications are incorporated herein by reference in their entirety. Other devices may also be used within the scope of this disclosure.

These and other implants may be employed, for example, as elements of an implant system, such as a spinal construct. In some embodiments, the implant system may be surgically introduced at site, such as a section of a spine, within a body of a patient. Surgical approaches include, for example, anterior lumbar interbody fusion (ALIF), direct lateral interbody fusion (DLIF), oblique lateral lumbar interbody fusion (OLLIF), oblique lateral interbody fusion (OLIF), transforaminal lumbar Interbody fusion (TLIF), posterior lumbar Interbody fusion (PLIF), various types of posterior or anterior fusion procedures, and/or any fusion or fixation procedure in any portion of the spinal column (sacral, lumbar, thoracic, and cervical), e.g., to restore the mechanical support function of one or more vertebrae.

The following discussion omits or only briefly describes certain components, features and functionality related to medical implants, installation tools, and associated surgical techniques, which are apparent to those of ordinary skill in the art. It is noted that various embodiments are described in detail with reference to the drawings, in which like reference numerals represent like parts and assemblies throughout the several views, where possible. Reference to various embodiments does not limit the scope of the claims appended hereto because the embodiments are examples of the inventive concepts described herein. Additionally, any example(s) set forth in this specification are intended to be non-limiting and set forth some of the many possible embodiments applicable to the appended claims. Further, particular features described herein can be used in combination with other described features in each of the various possible combinations and permutations unless the context or other statements clearly indicate otherwise.

Terms such as “same,” “equal,” “planar,” “coplanar,” “parallel,” “perpendicular,” etc. as used herein are intended to encompass a meaning of exactly the same while also including variations that may occur, for example, due to manufacturing processes. The term “substantially” may be used herein to emphasize this meaning, particularly when the described embodiment has the same or nearly the same functionality or characteristic, unless the context or other statements clearly indicate otherwise. The term “about” may encompass a meaning of being +/−10% of the stated value.

Unless otherwise specifically defined herein, all terms are to be given their broadest possible interpretation including meanings implied from the specification as well as meanings understood by those skilled in the art and/or as defined in dictionaries, treatises, etc. It must also be noted that, as used in the specification and the appended claims, the singular forms “a,” “an” and “the” include plural referents unless otherwise specified, and that the terms “comprises” and/or “comprising,” when used in this specification, specify the presence of stated features, elements, and/or components, but do not preclude the presence or addition of one or more other features, steps, operations, elements, components, and/or groups thereof.

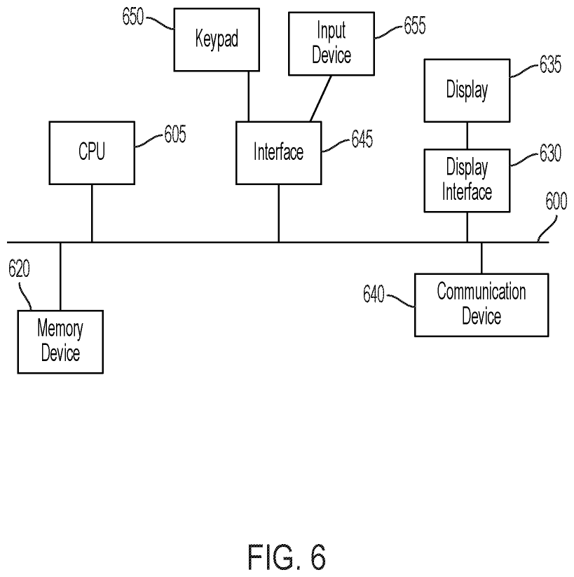

A “computing device”, “electronic device”, or “computer” refers a device or system that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions. Examples of electronic devices include personal computers, servers, mainframes, virtual machines, containers, mobile electronic devices such as smartphones, Internet-connected wearables, tablet computers, laptop computers, and appliances and other devices that can communicate in an Internet-of-things arrangement. In a client-server arrangement, the client device and the server are electronic devices, in which the server contains instructions and/or data that the client device accesses via one or more communications links in one or more communications networks. In a virtual machine arrangement, a server may be an electronic device, and each virtual machine or container also may be considered an electronic device. In the discussion below, a client device, server device, virtual machine or container may be referred to simply as a “device” for brevity. Additional elements that may be included in electronic devices will be discussed below in the context of FIG. 6.

The terms “memory,” “computer-readable medium” and “data store” each refer to a non-transitory device on which computer-readable data, programming instructions or both are stored. Unless the context specifically states that a single device is required or that multiple devices are required, the terms “memory,” “computer-readable medium” and “data store” include both the singular and plural embodiments, as well as portions of such devices such as memory sectors.

A “machine learning model” or a “model” refers to a set of algorithmic routines and parameters that can predict an output (or outputs) of a real-world process (e.g., prediction of an object trajectory, a diagnosis or treatment of a patient, a suitable recommendation based on a user search query, etc.) based on a set of input features, without being explicitly programmed. A structure of the software routines (e.g., number of subroutines and relation between them) and/or the values of the parameters can be determined in a training process, which can use actual results of the real-world process that is being modeled. Such systems or models are understood to be necessarily rooted in computer technology, and in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology.

A typical machine learning pipeline may include building a machine learning model from a sample dataset (referred to as a “training set”), potentially evaluating the model against one or more additional sample datasets (referred to as a “validation set” and/or a “test set”) to decide whether to keep the model and to benchmark how good the model is, and using the model in “production” to make predictions or decisions against live input data captured by an application service. Supervised learning, also known as supervised machine learning, is a subcategory of machine learning and artificial intelligence. Supervised learning is defined by its use of labeled datasets to train algorithms that classify data or predict outcomes accurately. As input data is fed into the supervised learning system, it may adjust model weights (or other parameters of an inferred function) until the model/function has been fitted appropriately to correctly determine class labels of new examples. Supervised learning helps solve for a variety of real-world problems at scale, such as classifying spam in a separate folder from the inbox. Example supervised learning algorithms include neural networks, such as Convolutional Neural Networks (CNNs).

Spinal constructs are generally designed to correct instability or deformity (or both) of the spinal column based on an understanding of the deformity or instability and the biomechanical forces acting on the pathologic alignment. Implants, such as pedicle screws, may be placed in the front (anterior) and/or the back (posterior) of the spine and may be joined by rods, plates, etc. The disc between the vertebrae is often removed and replaced with a bone graft or an interbody spacer. In some instances, the construct spans multiple levels of the spinal column and involves multiple vertebrae, e.g., to address the full extent of the pathology.

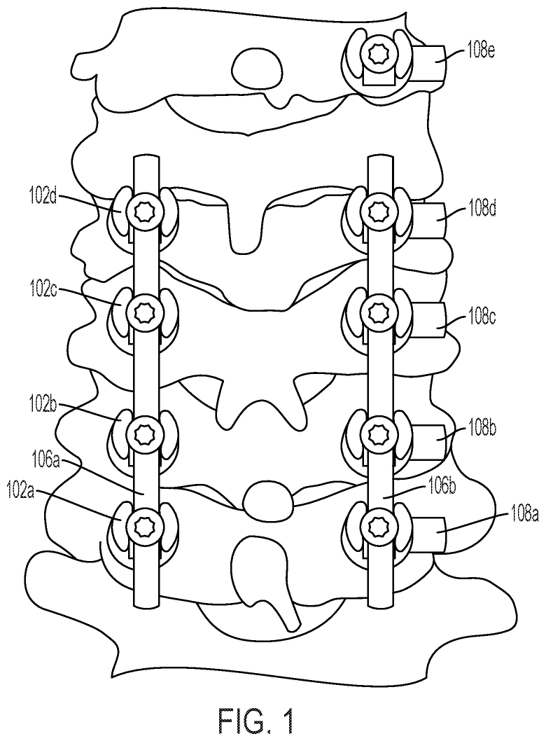

Referring to FIG. 1, an example multilevel spinal construct 100 is shown. FIG. 1 illustrates a total of five levels of the spinal column, four of which are included in (e.g., connected to) the spinal construct 100. In some embodiments, the spinal construct includes a greater or fewer number of spinal levels. A spinal fusion construct may include as few as two spinal levels. Such a spinal construct, configured to fuse two vertebrae together, is referred to as a single-level fusion. Spinal constructs may also include additional spinal levels and may be configured to fuse additional vertebrae. Both single-level and multilevel constructs are within the scope of this disclosure. The spinal construct 100 illustrated in FIG. 1 includes two spinal rods 106, 106a-b. Each spinal rod 106 is secured to four vertebrae. For example, rod 106a is secured to each of the four vertebrae by pedicle screws 102, 102a-d. In some embodiments, cortical screws may be used instead of pedicle screws. Rod 106b is similarly secured to the same four vertebrae. Thus, the example construct of FIG. 1 is a three-fusion-level construct.

As illustrated, each of the pedicle screws 102 of rod 106b includes an electronics housing 108, 108a-d configured to accommodate sensors and/or related electronics capable of providing telemetry related to the spinal construct 100, the spinal surgery/procedure, or other aspect or property of the patient 110. Each electronics housing 108 may include sensors or sensor systems configured to measure positions/orientations, forces, temperatures, or other parameters. Other examples of sensors may include, without limitation, pressure sensors, strain gauges, impedance sensors, temperature sensors, Inertial Measurement Units (IMUs), gyroscopes, magnetometers, and/or the like. The sensors may electrically interface with a processor or other logic device configured to acquire the sensor data and transmit the data, e.g., via a transceiver and antenna, to an external reader device. The pedicle screws (or other spinal implants) may also include a power source, such as a battery (rechargeable or otherwise) for powering the electronics. In some embodiments, each of the pedicle screws 102 of both spinal rods 106a, 106b include electronics housings 108. In other embodiments, select pedicle screws (or cortical screws) 102 of one or both rods 106 include electronics housings 108 and associated sensors. For example, sensors (and associated electronics) may be included on every pedicle screw (or cortical screw) 102 in the spinal construct 100 or may be distributed strategically across different pedicle screws 102 on the, e.g., left and right sides of the construct. Other configurations of spinal-construct components, electronics housings 108 and sensors are also within the scope of the disclosure.

Those skilled in the art will understand that the spinal construct 100 of FIG. 1 is one example and that other configurations are also possible. For example, spinal constructs 100 may have include more than two rods 106 or fewer than two rods 106. Spinal constructs 100 may have plates or other structures connecting pedicle screws 102 together. Spinal constructs 100 may include tethers or cords which interface with vertebrae, e.g., to apply a corrective force. Cords or tethers may be attached to vertebrae via screws or may pass around or through vertebrae (e.g., through holes drilled into parts of vertebrae). Spinal constructs 100 may include other implant devices, such as interbody cages or spacers or may consist entirely of interbody implants. All of these examples, and more, are also within the scope of this disclosure.

The vertebra at the highest level included in the construct 100 may be referred to as the Upper Instrumented Vertebra (UIV). As illustrated in FIG. 1, rod 106a is connected to the UIV via pedicle screw 102d. The other vertebrae included in the construct 100 may be referred to as UIV−1, UIV−2, and UIV−3, representing their positions relative to the UIV. As illustrated in FIG. 1, rod 106a is connected to UIV−1, UIV−2, and UIV−3 via pedicle screws 102c, 102b, and 102a, respectively. Similarly, the levels above the UIV may be referred to as UIV+1, UIV+2, and so forth. For simplicity, FIG. 1 shows one level above the UIV, i.e., UIV+1, which is the level adjacent to the UIV (and thus, the level adjacent to the construct 100). Because UIV+1 (and higher levels) are not included in (or connected to) the construct 100, the construct 100 itself does not transmit forces to these adjacent levels. However, levels adjacent to spinal fusion constructs 100 may be exposed to increased biomechanical forces due to the construct 100, particularly in cases involving long thoracolumbar deformity constructs 100 (e.g., those spanning four or more levels). These forces can lead to higher incidence of degeneration involving these adjacent levels and/or Proximal Junction Kyphosis (PJK) or Proximal Junction Failure (PJF). Research has shown that as many as 10-20% of long-construct spinal fusions result in some degree of adjacent-level breakdown over a period of ten years after surgery. In the absence of ongoing diagnostic, such as x-rays, the degeneration may go undetected until it becomes severe. With early detection, less intrusive therapies may be possible, including forms of physical therapy either with or without externally worn devices, such as a rigid brace or even a soft collar.

As shown in FIG. 1, pedicle screw of UIV+1 includes electronics housing 108e. Similar to the other electronics housings 108a-d, electronics housing 108e may also house sensors or sensor systems configured to measure parameters associated with UIV+1. However, the sensors or sensor systems housed in electronics housing 108e may be configured to measure different parameters than those housed in electronics housings 108a-d (i.e., those sensors associated with the spinal construct 100). Furthermore, electronics housing 108e may be secured to UIV+1 differently than how electronics housings 108a-d are secured to their respective vertebrae. For example, electronics housing 108e may be secured to UIV+1 using a screw having a relatively smaller shank (e.g., compared to pedicle screws 102a-102d). The smaller shank allows for subsequent replacement by a larger-shanked pedicle screw 102, e.g., to extend the construct 100 during a subsequent procedure to include that vertebra.

Including a smart spinal sensor at the vertebrae adjacent to the fusion construct 100 allows a surgeon (or other medical professional, such as a clinician) to assess kinematics and biomechanics of that level during the course of a post-operative period. The surgeon may be able to identify, e.g., adjacent level degeneration before the degeneration progresses to the point of requiring a surgical intervention. The assessment may be based on construct-related telemetry as well as telemetry from adjacent (or other) levels of the spinal column. That is, the assessment may be based on sensor information from sensor systems associated with the spinal construct 100 (or constructs) and sensor information from sensor systems associated with one or more levels adjacent to or otherwise unconnected to the spinal construct 100 (or constructs). In some examples, at least a portion of the sensor data is acquired while a patient 110 is performing a predefined assessment protocol. For instance, a clinician may ask the patient 110 to sit, stand, walk, bend over, rotate, turn, lie down or perform other activities while the clinician obtains sensor data from the construct sensors and from the implant sensors.

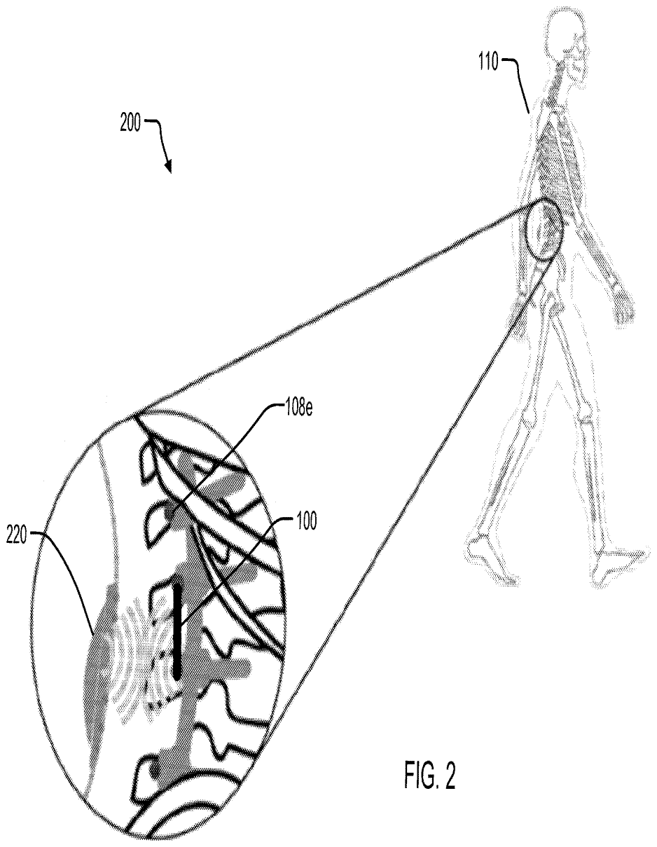

Referring to FIG. 2, an example monitoring system 200 is shown. The monitoring system 200 may include a spinal construct 100 and a wearable or external reader device 220. Referring back to FIG. 1, in various embodiments, the wearable reader device 220 may be in communication with one or more spinal implants. That is, the reader device 220 may include electronics systems, e.g., including one or more transceivers configured to receive telemetry from, e.g., sensor-equipped spinal implants. As illustrated, the spinal construct 100 is configured to provide telemetry to the reader device 220. That is, the spinal construct 100 may include one or more sensor systems configured to measure or sense properties related to the spinal construct 100 and provide the sensor data (e.g., wirelessly) to the reader device 220. Furthermore, one or more adjacent-level vertebra may also be configured to provide telemetry to the wearable reader device 220. As illustrated in FIG. 1, UIV+1 is configured to measure or sense properties related to the vertebral level that is adjacent to the construct 100 and provide the sensor data (e.g., wirelessly) to the reader device 220. Thus, telemetry related to the construct 100 and telemetry related to vertebral levels that are adjacent to (and not included in) the construct 100 are provided to the reader 220. Other telemetry related generally to the surgical sites, such as temperature readings, may also be provided to the reader 220 or measured by sensors included in the reader 220.

The reader device 220 is illustrated as being worn by the patient 110, e.g., against the patient's skin. For example, the wearable reader device 220 may be configured to be worn by the patient 110 across at least a portion of the patient's spine, such as a portion of the patient's lower back, a portion of the patient's middle back, a portion of the patient's upper back, etc. In this way, an electronics system, or a portion of an electronics system, of the reader 220 may be located close to the sources of telemetry (e.g., the spinal implants). The wearable reader device 220 may be secured to a patient 110 using an adhesive, or via one or more straps, braces, and/or the like. In some embodiments, a wearable reader device 220 may be part of a wearable garment such as, for example, a harness, a belt, a vest, a shirt, and/or the like. U.S. patent application Ser. No. 16/132,094, which is incorporated herein by reference in its entirety, describes example wearable electronic devices and systems which may be used within the scope of this disclosure.

The reader device 220 may also be separated from the patient 110 by distances that still allow wireless communication between the smart implants (e.g., of the spinal construct 100) and the reader device 220. In some examples, the reader device 220 is not a wearable device. For example, the reader device 220 may be a stand-alone device located in a clinician's examination room or area, or in the patient's home, e.g., where the patient 110 performs an assessment protocol under the guidance of the clinician (either in-person or remotely). In these cases and others, an external reader 220, such as the system disclosed in U.S. patent application Ser. No. 16/855,444, incorporated herein by reference in its entirety, may display or otherwise provide the telemetry to a medical professional for evaluation. The external reader 220 may also receive telemetry from other sources such as, but not limited to, one or more wearable sensor system that are affixed to the patient 110. The reader device 220 itself may also include one or more additional sensors. As discussed above, the combined sensor data may be obtained while the patient 110 performs an assessment protocol that may include bending, rotating, or other prescribed movement. Alternatively (or in addition), the combined data may be obtained, e.g., continually, during routine activities of the patient 110. Also as discussed above, a clinician (or other medical professional) may use the combined sensor data to assess the post-operative condition of the patient's spine. For example, a clinician may determine that PJK is likely based on one or more kinematic or biomechanical factors, such as the relative positions of the spinal construct 100 and the adjacent-level vertebra (e.g., UIV+1), or from the range of motion (or a change over time to the range of motion) between the spinal construct 100 and the adjacent-level vertebra (e.g., as the patient 110 performs the assessment protocol). Changes to these biomechanical factors may indicate the onset of a degradation. For example, if the height of the disc between UIV and UIV+1 decreases over time below a threshold height, or the range of motion between UIV and UIV+1 increases beyond an expected threshold.

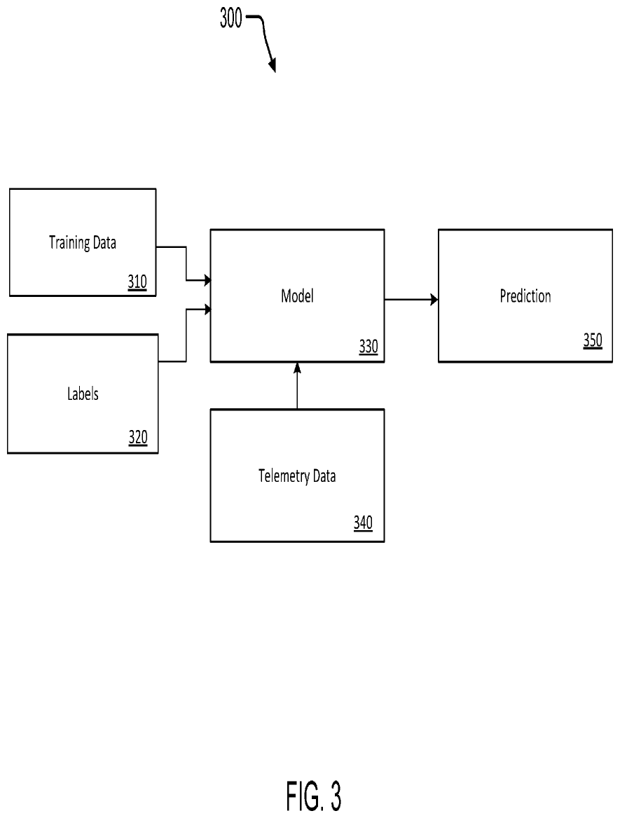

In some examples, a machine-learning system assesses the post-operative condition of the patient's spine. The machine-learning system may include a model 330 (FIG. 3) trained to predict the post-operative condition of the patient's spine, e.g., based on telemetry from spinal implants associated with a spinal construct 100 and telemetry from sensors associated with an adjacent-level vertebra (e.g., UIV+1). In some examples, the machine-learning model 330 further recommends a procedure to perform based on the predicted condition of the patient's spine. Possible predictions include: substantially no degeneration (and no procedure necessary), detectable degeneration (which may require more close or more frequent monitoring or beginning a non-intrusive intervention, such as wearing a soft collar), more substantial degeneration (which may require more intrusive, but non-surgical, intervention, such as wearing a body brace), and substantial degeneration (which may require surgical intervention to avoid failure).

FIG. 3 illustrates an example supervised machine-learning environment 300 for predicting the post-operative condition of the patient's spine and/or recommending a treatment for the condition, e.g., based on the combined sensor data discussed above (i.e., telemetry from a spinal construct 100 and telemetry from sensors associated with an adjacent-level vertebra). The telemetry data 340 (e.g., telemetered sensor data) may include the relative position (or other property) between the spinal construct 100 and UIV+1 (e.g., reflecting the height of the disc that separates the construct 100 from the adjacent vertebra); the range of motion of the UIV+1 with respect to the construct 100; the acceleration profile of UIV+1 with respect to the construct 100 (e.g., during normal activity of the patient 110 or when the patient 110 performs an assessment protocol); degree of possible flection/extension; temperature measurement (and/or temperature measurement variations at different locations on the construct 100); as well as changes over time to these and other sensor measurements. In some examples, the telemetry data 340 includes measurements related to additional vertebral levels, e.g., UIV+2, UIV+3, etc.

The environment 300 includes a model 330 which may be trained to predict conditions such as Proximal Junction Kyphosis (PJK) and/or Proximal Junction Failure (PJF) based on these telemetry data 340. In some examples, the model 330 is trained using training data 310 that is representative of the combined sensor data discussed above. A training supervisor may apply labels 320 to the training data 310 that indicate an associated condition. For example, the training data 310 may include post-operative telemetry data 340 from a large number of patients. The training supervisor may label 320 the training data 310 as, e.g., PJK, PJF, etc., based on whether those associated conditions are independently detected in the patient 110 (such as via x-ray images or other diagnostic). In this way, the model 330 may be trained to recognize features in the combined sensor data that are associated with PJK, PJF, and/or other conditions. After the model 330 has been trained, newly acquired telemetry data 340 may be applied to the model 330. The model 330 then classifies the telemetry data 340, based on its training, resulting in a prediction 350 of the condition of the patient's spine. In some examples, the model 330 is further trained to recommend a procedure to address the condition. Similar to how the model 330 is trained to recognize sensor data associated with particular conditions, the model 330 may also be trained to associate sensor data with appropriate corrective procedures. That is, a training supervisor may label training data 310 based on what procedure was used to successfully address the condition. In this way, the model 330 may be trained to recognize features in the telemetry data 340 that indicate a condition that is likely to be successfully addressed via a particular procedure.

In some examples, the model 330 is configured for continual training. For example, after applying telemetry data 340 to the model 330 to produce a predicted condition and/or a recommended procedure, the clinician may disagree with the model's output and may indicate the disagreement to the machine-learning system. In response, the machine-learning system may retrain the model 330 by adding (to the training data 310) the telemetry data 340 that resulted in disagreement and applying a label 320 (or labels) specified by the clinician to the newly added training data 310. In this way, the expertise of the clinician may be captured over time by the model 330. In some examples, the model 330 is cloud-based. That is, the model 330 may be remote from the reader device 220 and may be accessed via a network. In this case, a common model 330 may be continually trained by multiple clinicians, capturing the wisdom of multiple clinicians over time. In other examples, the model 330 is less frequently updated or may even be “locked” after initial training, e.g., to provide for consistent predictions over time.

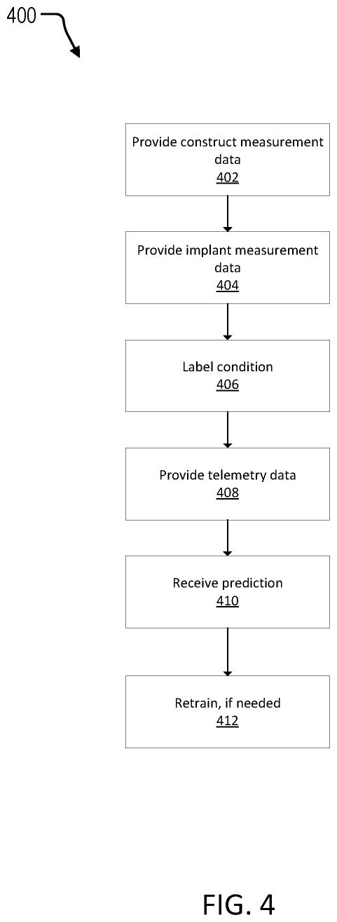

FIG. 4 illustrates a flow chart 400 of an example method of training a machine-learning model 330. At step 402, the method includes providing construct measurement data to the model 330 as training data 310. At step 404, the method includes providing implant measurement data to the model 330 as training data 310. At step 406, the method includes labeling the provided data to indicate an associated condition. For example, the training data 310 may be associated with patients who have experienced Proximal Joint Kyphosis. In these cases, the training supervisor may apply the label 320 “PJK” to the training data 310. Similarly, the training supervisor may apply the label 320 “Normal” to training data 310 associated with patients who have not experienced a pathology. By applying labels 320 to representative training data 310, the training supervisor trains the model 330 to effectively classify subsequently acquired telemetry data 340. At step 408, the method includes providing subsequently acquired telemetry data 340 to the trained model 330. Based on its training, the model 330 will predict a condition of the patient 110. At step 410, the training supervisor may review the model's prediction 350 and determine whether additional training is required. At step 412, the training supervisor may retrain the model 330, e.g., by applying an appropriate label to the subsequently acquired telemetry data 340.

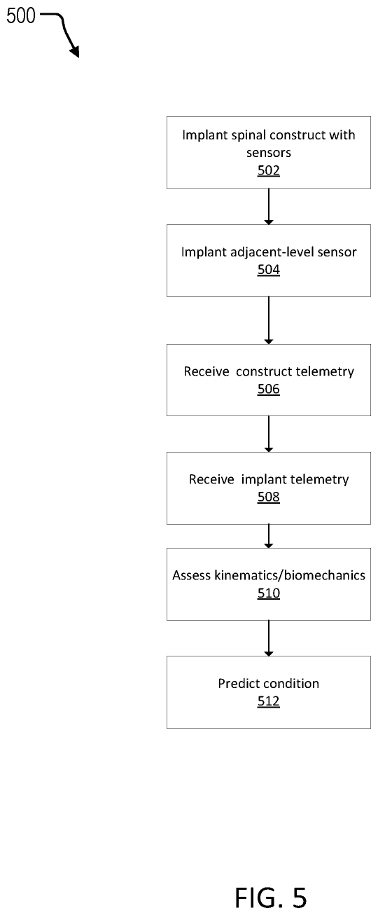

FIG. 5 illustrates a flow chart 500 of an example method of assessing a patient 110. At step 502, the method includes implanting a multilevel construct 100, such as a thoracolumbar deformity construct 100, spanning multiple (e.g., four or more) spinal levels. The multilevel construct 100 may be equipped with sensors to measure properties of the construct 100, the patient 110, the interface between the construct 100 and the patient 110, etc. For example, the sensors may measure the position of the construct 100 and/or forces applied to the construct 100 as the patient 110 moves. At step 504, the method incudes implanting a sensor to an adjacent-level vertebra. That is, implanting the sensor to a vertebra that is not included in the multilevel spinal construct 100, and it thus free to move relative to the spinal construct 100. In some examples, the adjacent-level sensor measures properties of the adjacent-level vertebra, such as position of the adjacent-level vertebra, which can be compared with position data related to the spinal construct 100. At steps 506 and 508, the method includes receiving measurement data (e.g., telemetry) from the construct 100 and the implant, respectively. At step 510, the method includes assessing one or more kinematic or biomechanical aspects of the construct 100 and implant, such as the relative positions of the construct 100 and implant (e.g., while the patient 110 performs assessment protocol movements), or the range of motion of the implant with respect to the construct 100. In some examples, the assessment includes a prediction 350 from a machine-learning model 330 which has been trained to predict a patient's condition, e.g., by recognizing patterns in associated telemetry data 340. At step 512, the method includes predicting the condition of the patient 110 based on the kinematic/biomechanical assessment and/or the output of the machine-learning model 330.

FIG. 6 illustrates example hardware that may be used to contain or implement program instructions. A bus 600 serves as the main information highway interconnecting the other illustrated components of the hardware. CPU 605 is the central processing unit of the system, performing calculations and logic operations required to execute a program. CPU 605, alone or in conjunction with one or more of the other elements disclosed in FIG. 6, is an example of a processor as such term is used within this disclosure. Read only memory (ROM) and random access memory (RAM) constitute examples of non-transitory computer-readable storage media 620, memory devices or data stores as such terms are used within this disclosure.

Program instructions, software or interactive modules for providing the interface and performing any querying or analysis associated with one or more data sets may be stored in the memory device 620. Optionally, the program instructions may be stored on a tangible, non-transitory computer-readable medium such as a compact disk, a digital disk, flash memory, a memory card, a USB drive, an optical disc storage medium and/or other recording medium.

An optional display interface 630 may permit information from the bus 600 to be displayed on the display 635 in audio, visual, graphic or alphanumeric format. Communication with external devices may occur using various communication ports 640. A communication port 640 may be attached to a communications network, such as the Internet or an intranet.

The hardware may also include an interface 645 which allows for receipt of data from input devices such as a keypad 650 or other input device 655 such as a touch screen, a remote control, a pointing device, a video input device and/or an audio input device.

The invention may be further described by reference to the following numbered clauses:

Clause 1. A method of generating a post-operative assessment of a patient, the method comprising:
obtaining one or more first measurements from one or more construct sensors internal to the patient, the one or more first measurements related to a property of a spinal construct of the patient, the spinal construct associated with at least one spinal level;
obtaining one or more second measurements from one or more implant sensors internal to the patient, the one or more second measurements related to a property of a spinal implant of the patient, the spinal implant associated with a different level than the at least one spinal level of the spinal construct; and determining, based on the one or more first measurements and the one or more second measurements, a condition of the patient.
   Clause 2. The method of clause 1, wherein:
the one or more construct sensors comprise at least one construct position sensor configured to provide a measurement of the position of the spinal construct;
the one or more implant sensors comprise at least one implant position sensor configured to provide a measurement of the position of the spinal implant; and
determining the condition of the patient comprises evaluating one or more biomechanical factors over time, the one or more biomechanical factors based on the measurement of the at least one construct position sensor and the measurement of the at least one implant position sensor.
   Clause 3. The method of clause 2, wherein at least one of the one or more biomechanical factors is a range of motion of the spinal implant relative to the spinal construct.
   Clause 4. The method of clause 1, wherein determining the condition of the patient comprises applying the one or more first measurements and the one or more second measurements to a machine-learning model trained, via supervised training, to predict the condition of the patient based on the one or more first measurements and the one or more second measurements.
   Clause 5. The method of clause 4, wherein:
the machine-learning model is further trained, via supervised training, to recommend a procedure to address the condition; and
the method further comprises applying the one or more first measurements and the one or more second measurements to the machine-learning model, causing the machine-learning model to recommend the procedure.
   Clause 6. The method of clause 4, wherein the machine-learning model is trained to predict Proximal Junction Kyphosis or Proximal Junction Failure.
   Clause 7. The method of clause 1, wherein:
the spinal construct comprises a multilevel spinal construct; and
the different level is adjacent to a level of the multilevel spinal construct.
   Clause 8. The method of clause 1, wherein the one or more construct sensors comprise at least one strain gauge.
   Clause 9. The method of clause 1, wherein at least one sensor comprises an impedance sensor configured to measure a status of a fusion process.
   Clause 10. The method of clause 1, wherein the spinal construct comprises a multilevel thoracolumbar deformity construct.
   Clause 11. A post-operative monitoring system comprising:
posterior spinal instrumentation associated with two or more vertebrae, the posterior spinal instrumentation comprising one or more spinal instrumentation sensors configured to measure a property of the posterior spinal instrumentation;
a spinal implant comprising one or more spinal implant sensors configured to measure a property of the spinal implant, the spinal implant associated with a different vertebra than the two or more vertebrae of the posterior spinal instrumentation;
a reader device configured to receive measurement data from the one or more spinal instrumentation sensors and the one or more spinal implant sensors; and
a patient assessment system configured to determine a condition of a patient based on the measurement data from the one or more spinal instrumentation sensors and the one or more spinal implant sensors.
   Clause 12. The post-operative monitoring system of clause 11, wherein:
the one or more spinal instrumentation sensors comprise at least one instrumentation position sensor configured to provide a measurement of the position of the posterior spinal instrumentation;
the one or more spinal implant sensors comprise at least one implant position sensor configured to provide a measurement of the position of the spinal implant; and
the patient assessment system is configured to determine the condition of the patient by evaluating one or more biomechanical factors over time, the one or more biomechanical factors based on the measurement of the at least one instrumentation position sensor and the measurement of the at least one implant position sensor.
   Clause 13. The post-operative monitoring system of clause 12, wherein at least one of the one or more biomechanical factors is a range of motion of the spinal implant relative to the posterior spinal instrumentation.
   Clause 14. The post-operative monitoring system of clause 11, wherein the patient assessment system is configured to determine the condition of the patient by applying the measurement data from the one or more spinal instrumentation sensors and the one or more spinal implant sensors to a machine-learning model trained, via supervised training, to predict the condition of the patient based on the measurement data from the one or more spinal instrumentation sensors and the one or more spinal implant sensors.
   Clause 15. The post-operative monitoring system of clause 14, wherein the machine-learning model is trained to predict Proximal Junction Kyphosis or Proximal Junction Failure.
   Clause 16. The post-operative monitoring system of clause 14, wherein:
the machine-learning model is further trained, via supervised training, to recommend a procedure to address the condition; and
the patient assessment system is further configured recommend the procedure based on the machine-learning model.
   Clause 17. The post-operative monitoring system of clause 11, wherein the different vertebra is adjacent to one of the two or more vertebrae.
   Clause 18. The post-operative monitoring system of clause 11, wherein the one or more spinal instrumentation sensors comprise at least one strain gauge.
   Clause 19. The post-operative monitoring system of clause 11, wherein the posterior spinal instrumentation comprises a multilevel thoracolumbar deformity construct.
   Clause 20. The post-operative monitoring system of clause 11, wherein at least one sensor comprises an impedance sensor configured to measure a status of a fusion process.

It will be appreciated that the various above-disclosed and other features and functions, or alternatives thereof, may be desirably combined into many other different systems or applications or combinations of systems and applications. Also that various presently unforeseen or unanticipated alternatives, modifications, variations or improvements therein may be subsequently made by those skilled in the art which are also intended to be encompassed by the following claims.

The breadth and scope of this disclosure should not be limited by any of the above-described example embodiments, but should be defined only in accordance with the following claims and their equivalents.

1-15. (canceled)

16. A method of generating a post-operative assessment of a patient, the method comprising:
obtaining one or more first measurements from one or more construct sensors internal to the patient, the one or more first measurements related to a property of a spinal construct of the patient, the spinal construct associated with at least one spinal level;
obtaining one or more second measurements from one or more implant sensors internal to the patient, the one or more second measurements related to a property of a spinal implant of the patient, the spinal implant associated with a different level than the at least one spinal level of the spinal construct; and
determining, based on the one or more first measurements and the one or more second measurements, a condition of the patient.

17. The method of claim 16, wherein:
the one or more construct sensors comprise at least one construct position sensor configured to provide a measurement of the position of the spinal construct;
the one or more implant sensors comprise at least one implant position sensor configured to provide a measurement of the position of the spinal implant; and
determining the condition of the patient comprises evaluating one or more biomechanical factors over time, the one or more biomechanical factors determined by the measurement of the at least one construct position sensor and the measurement of the at least one implant position sensor.

18. The method of claim 17, wherein at least one of the one or more biomechanical factors is a range of motion of the spinal implant relative to the spinal construct.

19. The method of claim 16, wherein determining the condition of the patient comprises applying the one or more first measurements and the one or more second measurements to a machine-learning model trained, via supervised training, to predict the condition of the patient based on the one or more first measurements and the one or more second measurements.

20. The method of claim 19, wherein:
the machine-learning model is further trained, via supervised training, to recommend a procedure to address the condition; and
the method further comprises applying the one or more first measurements and the one or more second measurements to the machine-learning model, causing the machine-learning model to recommend the procedure.

21. The method of claim 19, wherein the machine-learning model is trained to predict Proximal Junction Kyphosis or Proximal Junction Failure.

22. The method of claim 16, wherein:
the spinal construct comprises a multilevel spinal construct; and
the different level is adjacent to a level of the multilevel spinal construct.

23. The method of claim 16, wherein the one or more construct sensors comprise at least one strain gauge.

24. The method of claim 16, wherein at least one sensor comprises an impedance sensor configured to measure a status of a fusion process.

25. The method of claim 16, wherein the spinal construct comprises a multilevel thoracolumbar deformity construct.

26. A post-operative monitoring system comprising:
posterior spinal instrumentation associated with two or more vertebrae, the posterior spinal instrumentation comprising one or more spinal instrumentation sensors configured to measure a property of the posterior spinal instrumentation;
a spinal implant comprising one or more spinal implant sensors configured to measure a property of the spinal implant, the spinal implant associated with a different vertebra than the two or more vertebrae of the posterior spinal instrumentation;
a reader device configured to receive measurement data from the one or more spinal instrumentation sensors and the one or more spinal implant sensors; and
a patient assessment system configured to determine a condition of a patient based on the measurement data from the one or more spinal instrumentation sensors and the one or more spinal implant sensors.

27. The post-operative monitoring system of claim 26, wherein:
the one or more spinal instrumentation sensors comprise at least one instrumentation position sensor configured to provide a measurement of the position of the posterior spinal instrumentation;
the one or more spinal implant sensors comprise at least one implant position sensor configured to provide a measurement of the position of the spinal implant; and
the patient assessment system is configured to determine the condition of the patient by evaluating one or more biomechanical factors over time, the one or more biomechanical factors based on the measurement of the at least one instrumentation position sensor and the measurement of the at least one implant position sensor.

28. The post-operative monitoring system of claim 27, wherein at least one of the one or more biomechanical factors is a range of motion of the spinal implant relative to the posterior spinal instrumentation.

29. The post-operative monitoring system of claim 26, wherein the patient assessment system is configured to determine the condition of the patient by applying the measurement data from the one or more spinal instrumentation sensors and the one or more spinal implant sensors to a machine-learning model trained, via supervised training, to predict the condition of the patient based on the measurement data from the one or more spinal instrumentation sensors and the one or more spinal implant sensors.

30. The post-operative monitoring system of claim 29, wherein the machine-learning model is trained to predict Proximal Junction Kyphosis or Proximal Junction Failure.

31. The post-operative monitoring system of claim 29, wherein:
the machine-learning model is further trained, via supervised training, to recommend a procedure to address the condition; and
the patient assessment system is further configured to recommend the procedure based on the machine-learning model.

32. The post-operative monitoring system of claim 26, wherein the different vertebra is adjacent to one of the two or more vertebrae.

33. The post-operative monitoring system of claim 26, wherein the one or more spinal instrumentation sensors comprise at least one strain gauge.

34. The post-operative monitoring system of claim 26, wherein the posterior spinal instrumentation comprises a multilevel thoracolumbar deformity construct.

35. The post-operative monitoring system of claim 26, wherein at least one sensor comprises an impedance sensor configured to measure a status of a fusion process.
